(19) United States
(12) Patent Application Publication
(10) Pub. No.: US 2026/0279529 A1
(43) Pub. Date: Sep. 17, 2026

(54) REAL-TIME MEDICAL CODE VALIDATION USING CONTRASTIVE LEARNING AND RETRIEVAL-AUGMENTED GENERATION

(71) Applicant: Andrew Shirer, Riverview, FL (US)

(72) Inventor: Andrew Shirer, Riverview, FL (US)

(21) Appl. No.: 19/561,962

(22) Filed: Mar. 10, 2026

Related U.S. Application Data
(60) Provisional application No. 63/771,353, filed on Mar. 13, 2025.

(51) Int. Cl. G16H 15/00; G06N 20/00; G06Q 40/08

(52) U.S. Cl. CPC G16H 15/00; G06N 20/00; G06Q 40/08

(57) ABSTRACT

A system validates medical claim coding information during a coding workflow and before claim submission. The system receives diagnosis and procedure codes for a healthcare encounter and obtains embeddings produced by a contrastive machine-learning model trained on labeled valid and invalid code combinations. An embedding-based compatibility measure is determined using a similarity computation, and multi-code weighting generates an aggregated compatibility determination for encounters involving multiple codes. The aggregated determination is compared to a threshold to assign a preliminary classification of valid, invalid, or needs review. Policy or guidance passages are retrieved and used to confirm, revise, or override the preliminary classification, producing a final classification. A policy-cited explanation is generated from the retrieved passages. The system outputs the final classification and explanation to a user interface, enabling correction of potential coding errors before claim submission.

### CROSS-REFERENCE TO RELATED APPLICATIONS

This application claims the benefit of U.S. Provisional Application No. 63/771,353, filed Mar. 13, 2025, entitled “REAL-TIME MEDICAL CODE VALIDATION USING CONTRASTIVE LEARNING AND RETRIEVAL-AUGMENTED GENERATION,” the entire disclosure of which is incorporated herein by reference.

### FIELD OF THE INVENTION

The present disclosure relates to healthcare coding and billing systems, and more particularly to a computer-implemented system and method for real-time validation of medical claim coding information using contrastive machine-learning embeddings and retrieval-augmented generation of policy-cited explanations.

### BACKGROUND OF THE INVENTION

Medical coding and billing serve as the bridge between clinical care and financial reimbursement in the healthcare industry. The coding process involves translating medical diagnoses, procedures, and equipment into standardized alphanumeric codes drawn from multiple code sets, including ICD-10-CM (International Classification of Diseases, Tenth Revision, Clinical Modification) for diagnoses, CPT (Current Procedural Terminology) and HCPCS (Healthcare Common Procedure Coding System) for outpatient procedures, and ICD-10-PCS (International Classification of Diseases, Tenth Revision, Procedure Coding System) for inpatient procedures.

The complexity of these coding systems creates persistent challenges for healthcare organizations. Annual or quarterly updates to multiple code sets burden provider billing teams and create opportunities for error. According to industry estimates, U.S. healthcare systems spend upwards of $266 billion per year on administrative waste, with claim denials and rework among the major drivers. Up to 20% of all medical claims face initial denial, leading to rework costs of $25 to $118 per claim. In FY2024, the Centers for Medicare and Medicaid Services (CMS) documented more than $50 billion in improper Medicare payments, largely due to missing documentation and avoidable coding errors.

A critical shortcoming of existing approaches is that coding errors are typically discovered only after claim submission, when the payer issues a denial notice. This post-submission detection model forces providers into costly rework cycles of appeals and resubmissions, with delays of weeks or months before revenue is recovered.

Existing tools for diagnosis-procedure validation fall into two broad categories: (1) post-submission editors that use static, manually curated rule sets to identify coding errors after claims are generated, and (2) AI-based auto-coding systems that use natural language processing (NLP) to extract codes from clinical documentation.

Post-submission editors, such as rule-based computer-assisted coding (CAC) systems, rely on predefined rules that must be manually updated to remain in sync with evolving payer policies. These static rules often fail to incorporate payer-specific coverage determinations and cannot dynamically adapt to newly emerging code combinations or guideline changes. Their quarterly or annual update cycles create windows during which the system operates with outdated information.

AI-based auto-coding systems focus primarily on assigning codes from free-text clinical documentation rather than validating whether a proposed diagnosis-procedure combination is compatible. These systems typically handle single-diagnosis, single-procedure encounters but struggle with the multi-code complexity of inpatient or multi-specialty billing scenarios. Furthermore, most AI auto-coders operate as “black boxes,” failing to provide transparent, policy-grounded justifications for their recommendations.

Neither category of existing solution validates diagnosis-procedure code combinations in real time at the point of coding, provides multi-code weighted assessment for complex encounters, or generates policy-cited explanations grounded in current authoritative guidelines. There is accordingly a need for systems and methods that address these deficiencies.

### SUMMARY OF THE INVENTION

The present disclosure provides systems, methods, and computer-readable media for validating medical claim coding information in real time during a coding workflow and before claim submission.

According to a first aspect, a computer-implemented system for validating medical claim coding information includes a memory storing executable instructions and one or more data stores including medical code data and policy guidance data, and one or more processors configured to execute the instructions. The processors receive a set of medical codes including at least one diagnosis code and at least one procedure code associated with a healthcare encounter during a coding workflow and before claim submission. The processors obtain embeddings corresponding to the medical codes, the embeddings having been produced by a contrastive machine-learning model trained on labeled valid and invalid diagnosis-procedure code combinations. The processors determine an embedding-based compatibility measure based on a similarity computation applied to the embeddings, apply multi-code weighting to generate an aggregated compatibility determination, compare the aggregated determination to at least one threshold to assign a preliminary classification indicating valid, invalid, or needs review, retrieve policy or guidance passages relevant to the medical codes, confirm, revise, or override the preliminary classification based on the retrieved passages to produce a final classification, generate an explanation associated with the final classification based on the retrieved passages, and output the final classification and the explanation to a user interface during the coding workflow.

According to a second aspect, a computer-implemented method for validating medical claim coding information includes receiving encounter-specific diagnosis and procedure codes, obtaining embeddings from a contrastive machine-learning model, determining a similarity-based compatibility measure, aggregating the compatibility measure using multi-code weighting logic, assigning a preliminary classification based on threshold comparison, retrieving current coding guidance passages, confirming or revising the preliminary classification based on the guidance passages to produce a final classification, generating a policy-backed explanation, and presenting the final classification together with the explanation before claim submission.

According to a third aspect, a non-transitory computer-readable medium stores instructions that, when executed, cause one or more processors to perform operations including receiving diagnosis and procedure code information during a point-of-coding workflow, obtaining embeddings from a contrastive machine-learning model, determining an embedding-based compatibility measure, applying multi-code weighting, comparing to a threshold to assign a preliminary classification, retrieving policy guidance, confirming or revising the preliminary classification to produce a final classification, generating a policy-cited explanation, and outputting the final classification and explanation before claim submission.

The systems and methods disclosed herein may provide one or more of the following advantages: (a) real-time validation at the point of coding rather than post-submission detection, (b) learned embedding-based compatibility assessment that captures nuanced billing patterns beyond static rules, (c) multi-code weighted analysis that handles complex encounters with multiple diagnoses and procedures, (d) transparent, policy-cited explanations grounded in authoritative guidelines, and (e) a two-step validation flow that balances computational efficiency with thoroughness by applying threshold-based classification followed by retrieval-augmented confirmation or override.

### BRIEF DESCRIPTION OF THE DRAWINGS

A further understanding of the nature and advantages of particular embodiments may be realized by reference to the remaining portions of the specification and the drawings, in which like reference numerals are used to refer to similar components.

FIG. 1 illustrates a high-level system architecture diagram showing an offline training pipeline and an online real-time validation pipeline for a medical code validation system, according to aspects of the present disclosure.

FIG. 2 illustrates a flowchart of a two-step validation and explanation flow, including threshold-based preliminary classification followed by retrieval-augmented confirmation or override, according to aspects of the present disclosure.

FIG. 3 illustrates a block diagram of multi-code weighting logic for encounter-level classification, showing aggregation of individual compatibility measures across primary and secondary diagnosis codes, according to aspects of the present disclosure.

FIG. 4 illustrates a block diagram of a contrastive fine-tuning training pipeline using a two-tower architecture, showing separate processing towers for diagnosis codes and procedure codes, according to aspects of the present disclosure.

FIG. 5 illustrates a system integration architecture diagram showing deployment of the validation system as a containerized microservice with network-accessible interfaces for integration with electronic health record systems, practice management systems, and revenue cycle management platforms, according to aspects of the present disclosure.

### DETAILED DESCRIPTION OF PREFERRED EMBODIMENTS

While various aspects and features of certain embodiments have been summarized above, the following detailed description illustrates exemplary embodiments in further detail to enable one skilled in the art to practice such embodiments. The described examples are provided for illustrative purposes and are not intended to limit the scope of the invention. In the following description, numerous specific details are set forth in order to provide a thorough understanding of the described embodiments. It will be apparent to one skilled in the art, however, that other embodiments of the present invention may be practiced without some of these specific details. Several embodiments are described herein, and while various features are ascribed to different embodiments, it should be appreciated that the features described with respect to one embodiment may be incorporated with other embodiments as well.

As used herein, the terms “or” and “and/or” are to be interpreted as inclusive or meaning any one or any combination. The use of the term “including,” as well as other forms such as “includes” and “included,” should be considered non-exclusive. Terms such as “element” or “component” encompass both elements and components including one unit and elements and components that include more than one unit, unless specifically stated otherwise.

### Section A: Core Validation Engine

### A.1 System Overview

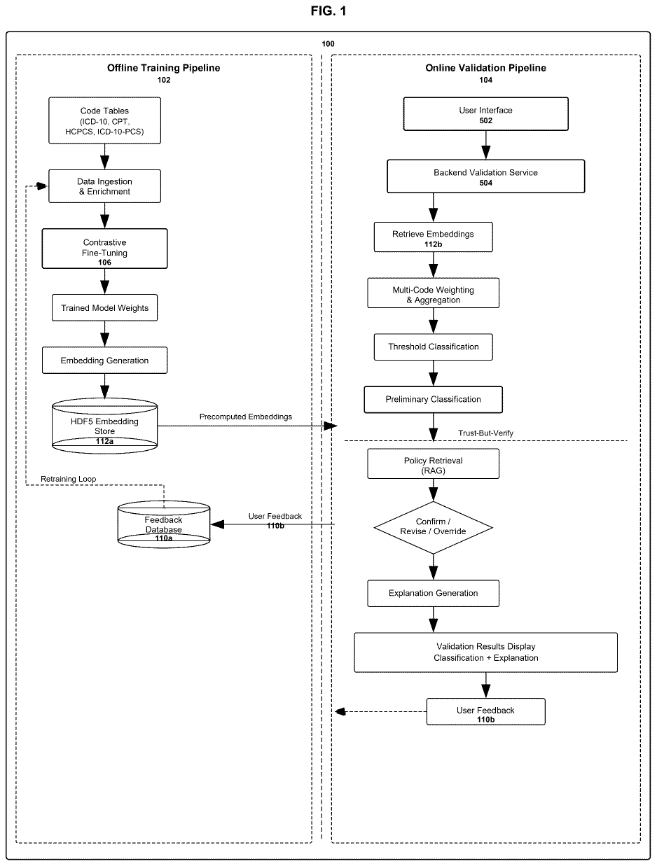

Referring now to FIG. 1, a medical code validation system 100 according to an embodiment of the present disclosure includes an offline training pipeline 102 and an online real-time validation pipeline 104. The offline training pipeline 102 produces trained model artifacts that the online real-time validation pipeline 104 uses to perform real-time validation of medical code combinations during a coding workflow and before claim submission.

The system 100 includes a memory storing executable instructions and one or more data stores including medical code data and policy guidance data. The medical code data may include code tables from one or more of ICD-10-CM, ICD-10-PCS, CPT, and HCPCS coding systems. The policy guidance data may include indexed documents from authoritative sources such as CMS, the National Correct Coding Initiative (NCCI), payer-specific guidelines, and internal compliance guidance.

One or more processors are configured to execute the instructions to perform the validation operations described herein. The system 100 receives, during a coding workflow and before claim submission, a set of medical codes including at least one diagnosis code and at least one procedure code associated with a healthcare encounter.

### A.2 Contrastive Machine-Learning Model and Two-Tower Architecture

The system 100 employs a contrastive machine-learning model 106 to produce embeddings for medical codes. In a preferred embodiment, the contrastive machine-learning model 106 comprises a two-tower architecture 108, also referred to as a Siamese model architecture, in which a first tower 108a generates embeddings for diagnosis codes and a second tower 108b generates embeddings for procedure codes.

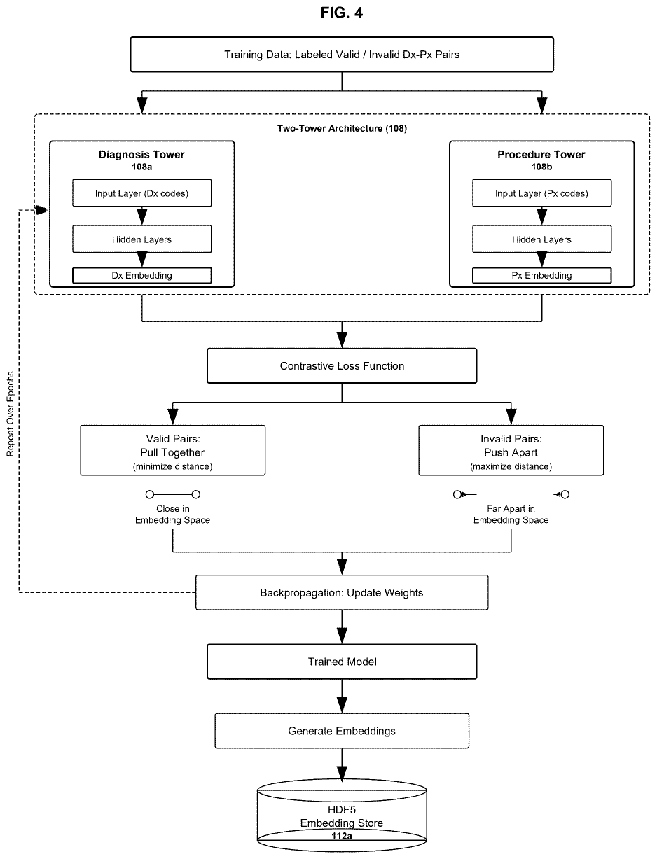

Referring also to FIG. 4, the two-tower architecture 108 is trained using a contrastive loss function that reduces the distance between embeddings of valid diagnosis-procedure code combinations and increases the distance between embeddings of invalid diagnosis-procedure code combinations. During training, the contrastive loss function encourages the model to “pull together” embeddings of valid code pairs in the embedding space while “pushing apart” embeddings of invalid code pairs. This contrastive learning approach enables the model to capture subtle patterns and relationships between diagnosis and procedure codes that may not be apparent in traditional rule-based systems.

The contrastive fine-tuning process involves several steps. The system prepares training data by assembling pairs of diagnosis and procedure codes along with their validity labels. Each pair is derived from audited real-world coding and billing scenarios and is associated with references to official coding guidance from one or more authoritative sources, such as CMS, NCCI, and payer-specific guidelines. The system feeds these pairs through the two-tower architecture 108, computing embeddings for each code in the pair. The system then applies the contrastive loss function to evaluate the similarity between the embeddings of each pair. For valid pairs, the loss function encourages the embeddings to be closer together. For invalid pairs, the loss function pushes the embeddings further apart. The system uses backpropagation to update the weights of the neural networks in both towers, gradually refining the model's ability to distinguish between valid and invalid code combinations. This process is repeated over multiple training epochs until the model achieves satisfactory performance on a validation dataset.

In initial validation tests, the contrastive embedding model achieved an F1 score of 0.8710, ROC-AUC of 0.9430, Precision of 0.8319, and Recall of 0.8813 at a recommended similarity threshold of 0.80.

### A.3 Embedding Generation and Storage

Once the contrastive fine-tuning process is complete, the system uses the trained model to generate fine-tuned embeddings for all relevant medical codes. These embeddings capture the learned relationships between codes, encoding information about their validity when used in combination.

In a preferred embodiment, the embeddings are precomputed during an offline training phase and stored in a format enabling retrieval during real-time validation without re-executing the contrastive machine-learning model for each validation request. This precomputation approach ensures real-time responses without re-running expensive model inferences for every validation request.

In one embodiment, the precomputed embeddings are stored in an HDF5-based storage format (Hierarchical Data Format version 5). The HDF5 format enables efficient storage and rapid retrieval of large arrays of embedding vectors, supporting sub-second lookup times even for large code sets encompassing tens of thousands of medical codes.

In some embodiments, the system employs approximate nearest neighbor (ANN) indexing to accelerate retrieval of the embeddings, retrieval of policy or guidance passages, or both. The ANN indexing creates an index structure that organizes code embeddings in a way that facilitates rapid similarity searches. Different ANN indexing strategies may be employed for different components of the validation process. For example, one index may be used for the initial embedding-based similarity computation, while another may be used for retrieving relevant policy documents during the explanation generation phase.

### A.4 Similarity Computation and Compatibility Measure

During real-time validation, the system obtains embeddings corresponding to the received medical codes. In embodiments where embeddings are precomputed, the system retrieves the embeddings from the precomputed embedding store. In other embodiments, the system may generate embeddings on demand using the trained contrastive model.

The system determines an embedding-based compatibility measure for the set of medical codes based at least in part on a similarity computation applied to the embeddings. In a preferred embodiment, the similarity computation comprises a cosine similarity calculation between the embedding vectors of the diagnosis code and the procedure code. Other similarity measures, such as Euclidean distance or dot product similarity, may be used in alternative embodiments.

### A.5 Multi-Code Weighting

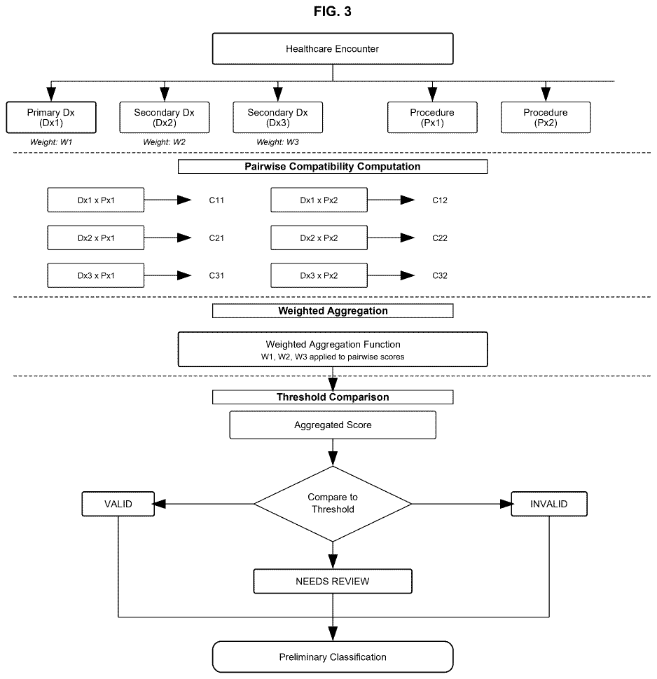

Referring also to FIG. 3, for encounters involving multiple diagnosis codes, multiple procedure codes, or both, the system applies multi-code weighting to generate an aggregated compatibility determination for the encounter. The multi-code weighting logic assigns different weights to primary and secondary diagnosis codes when generating the aggregated compatibility determination.

In a preferred embodiment, the system computes individual compatibility measures for respective diagnosis-procedure code pairings within the encounter, then generates a single aggregated compatibility score from the individual measures. For example, the system may compute a weighted similarity score that assigns higher weight to the primary diagnosis code and lower weights to secondary diagnosis codes, reflecting the clinical and billing significance hierarchy.

The multi-code weighting logic may incorporate domain-specific knowledge about common code combinations and clinical scenarios. For example, the system may assign higher weights to certain combinations of codes that frequently occur together in specific medical specialties or for particular types of encounters. The weighting logic factors in both primary and secondary codes to capture the true complexity of hospital billing, including complex inpatient encounters with multiple procedures and comorbidities.

### A.6 Threshold Classification and Three-Class Output

The system compares the aggregated compatibility determination to at least one threshold to assign a preliminary classification indicating valid, invalid, or needs review. In a preferred embodiment, the preliminary classification comprises a three-class output.

The “valid” classification is assigned when the aggregated compatibility determination exceeds the threshold, indicating that the code combination is likely compatible. The “invalid” classification is assigned when the aggregated compatibility determination falls below a lower threshold, indicating a likely mismatch. The “needs review” classification is assigned when the aggregated compatibility determination falls within a predefined threshold band, or when retrieved policy or guidance passages conflict with the preliminary classification.

In one embodiment, a similarity threshold of 0.80 is used as a baseline, with the needs review band encompassing a predefined range around this threshold. The threshold may be calibrated over time based on coding specialties and denial patterns in the deployment environment.

The needs review classification serves as a signal for human coders or supervisors to perform additional scrutiny. The system may provide additional context or explanations along with the flag to guide the review process. This approach balances automation with human expertise, ensuring that complex or borderline cases receive appropriate attention.

### Section B: Retrieval-Augmented Explanation Engine

### B.1 Two-Step Validation Flow

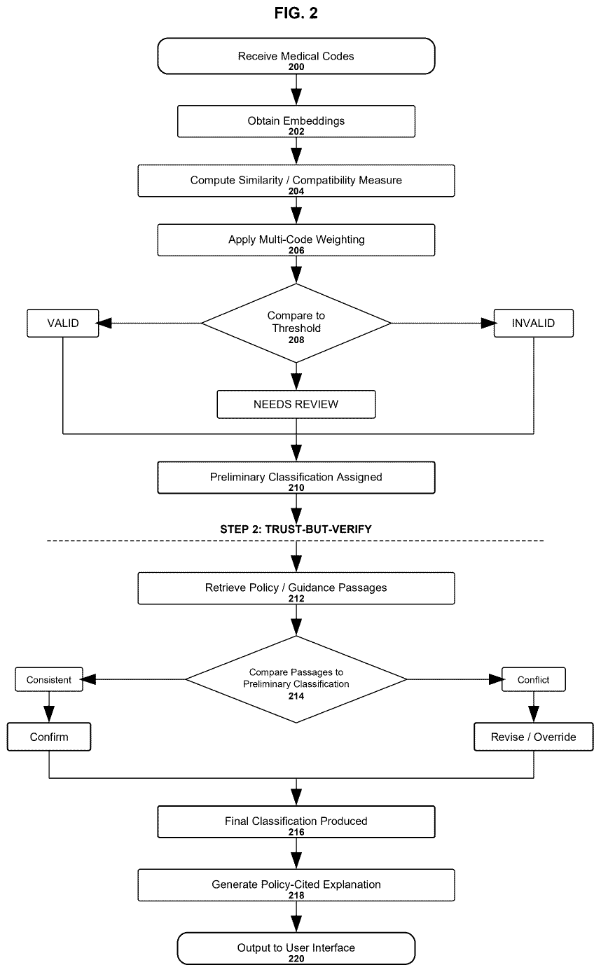

Referring now to FIG. 2, the system employs a two-step validation process to produce a final classification. In the first step, the system applies the threshold-based classification described in Section A.6 to assign the preliminary classification. In the second step, the system retrieves policy or guidance passages and uses them to confirm, revise, or override the preliminary classification.

This two-step approach, referred to herein as a “trust-but-verify” flow, balances computational efficiency with thoroughness. The threshold-based first step provides rapid initial assessment, while the retrieval-augmented second step ensures that the classification is consistent with current authoritative guidelines.

The final classification is produced by the one or more processors confirming the preliminary classification when the retrieved policy or guidance passages are consistent with the preliminary classification, and revising or overriding the preliminary classification when the retrieved policy or guidance passages conflict with the preliminary classification. For example, if the threshold-based classification indicates “valid” but the retrieved policy passages indicate that the code combination violates a recently updated NCCI bundling rule, the system may override the preliminary classification to “invalid” or “needs review.”

### B.2 Policy Retrieval and Citation

The system retrieves one or more policy or guidance passages relevant to the set of medical codes. The policy or guidance passages may include at least one of: payer-specific guidance, guidance from the Centers for Medicare and Medicaid Services (CMS), guidance from the National Correct Coding Initiative (NCCI), or internal compliance guidance.

In a preferred embodiment, the policy guidance data is maintained in a retrieval index that is updated to reflect changes in coding rules, payer rules, or regulatory guidance. The retrieval index may be updated periodically or in response to published guideline changes, ensuring that the system always references current authoritative information. The system retrieves the relevant passages from this updated retrieval index.

The retrieval mechanism may use techniques such as semantic search or embedding-based similarity to identify the most pertinent policy documents or sections. In one embodiment, retrieving the current coding guidance passages is performed by querying an indexed collection of coding policies and guidelines to identify relevant guidance passages, and providing the identified passages as context for generating the policy-backed explanation.

### B.3 Explanation Generation

The system generates an explanation associated with the final classification based on the retrieved policy or guidance passages. In a preferred embodiment, the explanation is generated using a specialized language model that receives the retrieved policy passages as context along with the code validation information.

The explanation may include: (a) a summary of the final classification (valid, invalid, or needs review); (b) a brief rationale for the classification outcome; (c) one or more citations to specific policy or guidance passages from which the explanation was generated; and (d) any additional context or considerations important for the coder to understand.

The inclusion of specific policy citations in explanations facilitates auditing processes and helps demonstrate compliance with coding standards. The policy-cited explanations may also serve an educational function, helping coders improve their understanding of coding guidelines over time.

The system may tailor the level of detail in the explanations based on user preferences or the complexity of the coding scenario. For straightforward validations, the system may provide brief, high-level explanations. For complex or ambiguous cases, the system may generate more detailed explanations with additional policy citations and context.

### B.4 User Feedback and Retraining

In some embodiments, the system is further configured to receive user feedback regarding a classification result and store the user feedback in a feedback data store for subsequent use in refining the contrastive machine-learning model or the policy guidance data. Users may submit feedback through the system interface, reporting discrepancies or issues they encounter during the coding process.

In a preferred embodiment, the stored user feedback is incorporated into a periodic retraining pipeline that updates the contrastive machine-learning model, the embeddings, or both based at least in part on the stored user feedback. The periodic retraining pipeline may analyze accumulated feedback to identify patterns or recurring issues, and use this analysis to inform updates to the validation logic, embedding models, or explanation generation processes.

This feedback mechanism enables the system to continuously refine its validation logic and improve its performance over time. By analyzing patterns in user feedback and final coding decisions, the system may adapt its thresholds and weighting logic to improve accuracy and relevance.

### Section C: Deployment and Integration

### C.1 System Architecture

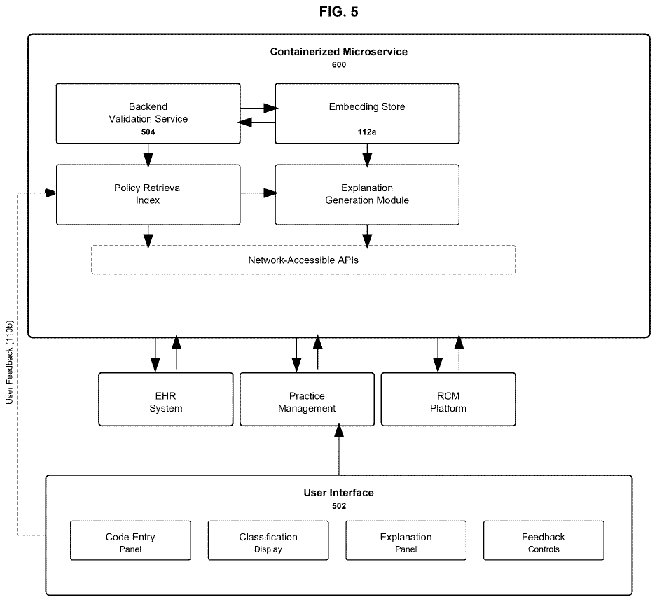

Referring now to FIG. 5, in some embodiments the system comprises a front-end user interface component 502 and a backend validation service 504 exposed through one or more network-accessible application programming interfaces (APIs). The front-end user interface component 502 is configured to receive the set of medical codes from a user and to display the final classification and the explanation to the user.

The front-end user interface component 502 interfaces with users through two primary interaction paths: a user start flow where encounter details and procedure codes are entered, and a user end flow where the final classification and explanation information are displayed. The system incorporates a user feedback loop that enables users to provide feedback on the validation results through the user interface.

The backend validation service 504 performs the multi-code weighting, threshold classification, policy retrieval, and explanation generation operations described herein. Communication between the front-end component 502 and the backend service 504 occurs through network-accessible API requests and responses.

### C.2 Containerized Microservice Deployment

In some embodiments, the system is deployed as a containerized microservice configured for integration with one or more of: an electronic health record (EHR) system, a practice management system, or a revenue cycle management (RCM) platform. The containerized microservice architecture allows the system to be deployed on-premises or in cloud environments, depending on the specific needs and infrastructure of healthcare organizations.

The system may expose network-accessible API endpoints to facilitate integration with other healthcare software systems. These endpoints may support operations including submitting code combinations for validation, retrieving validation results, and accessing policy-cited explanations. In some embodiments, the endpoints may support batch processing of multiple code combinations.

The containerized microservice may be designed to scale horizontally, allowing healthcare organizations to adjust the system's capacity based on validation volume and performance requirements. The containerized architecture offers portability across different computing environments, isolation from the host system and other applications, simplified version management and update processes, and efficient use of computing resources.

The system may include monitoring and logging capabilities to track performance and usage of the validation service. These features may assist in troubleshooting, capacity planning, and ensuring compliance with healthcare regulations.

### Section D: Additional Embodiments

The following additional embodiments are described to illustrate extensions and alternative implementations of the systems and methods disclosed herein.

### D.1 Multi-Agent Orchestration Architecture

In some embodiments, the validation system may employ a multi-agent orchestration architecture in which a directed acyclic graph (DAG) of specialist agents collaborates to produce a comprehensive defensibility assessment for a healthcare encounter. Each specialist agent may be responsible for a distinct aspect of the validation process, such as clinical criteria evaluation, evidence extraction, diagnosis validation, or financial impact analysis.

The orchestration engine may manage agent execution order, data dependencies between agents, and aggregation of individual agent outputs into a unified assessment. Each agent may operate according to a defined contract specifying its required inputs, expected outputs, and execution constraints. The DAG structure ensures that agents execute in a correct dependency order while allowing independent agents to execute concurrently for improved throughput.

In some embodiments, the specialist agents may include one or more of: a clinical criteria agent that evaluates whether clinical documentation supports each diagnosis code against published clinical criteria; an evidence extraction agent that identifies and extracts relevant clinical evidence from the patient record; a diagnosis validation agent that validates individual diagnosis codes against clinical criteria and documentation; a financial impact agent that estimates the monetary impact of potential coding changes; and a DRG (Diagnosis-Related Group) grouper agent that determines the appropriate DRG classification based on the diagnosis and procedure codes.

### D.2 Adversarial Critic and Revision Cycle

In some embodiments, the system may incorporate an adversarial critic and revision cycle in which a rationale builder agent generates a structured rationale for a validation determination, and an adversarial critic agent evaluates the rationale for logical gaps, unsupported assertions, or policy misalignment. If the adversarial critic identifies deficiencies, the rationale builder may revise its output in response to the critic's findings, and the critic may re-evaluate the revised rationale. This iterative cycle may continue for a configurable number of iterations or until the critic determines that the rationale is satisfactory.

The adversarial revision cycle may improve the quality and defensibility of validation rationales by subjecting them to structured scrutiny before they are finalized. The critic agent may evaluate rationales against criteria including factual accuracy, logical coherence, completeness of evidence citations, and alignment with applicable coding policies and guidelines.

### D.3 DRG Vulnerability Analysis and Financial Impact Modeling

In some embodiments directed to inpatient hospital encounters, the system may perform DRG vulnerability analysis. This analysis may involve determining the current DRG assignment based on the coded diagnoses and procedures, identifying diagnoses that contribute to severity classification (such as Major Complication/Comorbidity or Complication/Comorbidity status), and simulating the financial impact of potential coding changes.

The financial impact modeling may estimate the reimbursement difference between the current DRG assignment and alternative DRG assignments that would result from adding, removing, or modifying specific diagnosis or procedure codes. This analysis may help prioritize which coding decisions have the greatest financial impact and warrant the most careful review.

### D.4 Evidence Provenance and Audit-Grade Output Structures

In some embodiments, every evidence span extracted from the clinical record may carry a content hash and a citation to its source section, enabling verification that the evidence has not been altered or taken out of context. The system may employ deterministic extraction methods for structured data elements such as laboratory values, vital signs, and medication records, reserving language model-based extraction for unstructured clinical narratives.

The system output structures may be defined using typed data contracts that enforce schema validation, ensuring that all outputs conform to a predetermined format suitable for audit review. These audit-grade output structures may be designed to function as defensible workpapers that could be presented in response to a payer audit or regulatory inquiry.

In some embodiments, the system may apply protected health information (PHI) de-identification at multiple processing layers, role-based access controls governing which users may view PHI-bearing evidence, and audit logging for all data access events with correlation identifiers linking related operations.

### D.5 FHIR-Based Data Intake

In some embodiments, the system may accept healthcare encounter data formatted according to the HL7 FHIR (Fast Healthcare Interoperability Resources) standard. FHIR-formatted data may be transformed into the system's internal representation for processing through the validation pipeline described herein.

### General Implementation Notes

The methods and systems described herein may be implemented using one or more computing systems including tangible computer-readable storage media that hold machine-readable instructions executable by one or more processors. The computing systems may include memory devices, display subsystems for rendering graphical user interfaces, input subsystems for receiving user input, and communication subsystems for networked communication.

The described methods may be executed, provided, or implemented for a user or one or more computing devices via a computer-program product such as an application programming interface (API). The computing system may be configured to communicate with a plurality of computing devices over a computer network.

Since many modifications, variations, and changes in detail can be made to the described embodiments of the invention, it is intended that all matters in the foregoing description and shown in the accompanying drawings be interpreted as illustrative and not in a limiting sense. The scope of the invention should be determined by the appended claims and their legal equivalents.

In addition, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. It is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

What is claimed is:

1. A computer-implemented system for validating medical claim coding information, comprising: a memory storing executable instructions and one or more data stores including medical code data and policy guidance data; and one or more processors configured to execute the instructions to: receive, during a coding workflow and before claim submission, a set of medical codes including at least one diagnosis code and at least one procedure code associated with a healthcare encounter; obtain embeddings corresponding to the medical codes, the embeddings having been produced by a contrastive machine-learning model trained on labeled valid and invalid diagnosis-procedure code combinations; determine an embedding-based compatibility measure for the set of medical codes based at least in part on a similarity computation applied to the embeddings; apply multi-code weighting to the set of medical codes to generate an aggregated compatibility determination for the encounter; compare the aggregated compatibility determination to at least one threshold to assign a preliminary classification indicating valid, invalid, or needs review; retrieve one or more policy or guidance passages relevant to the set of medical codes; confirm, revise, or override the preliminary classification based on the retrieved one or more policy or guidance passages to produce a final classification;
generate an explanation associated with the final classification based on the retrieved one or more policy or guidance passages; and output, to a user interface, the final classification and the explanation during the coding workflow.

2. A computer-implemented method for validating medical claim coding information, comprising: receiving, during a coding workflow and before claim submission, encounter-specific diagnosis and procedure codes associated with a healthcare encounter; obtaining embeddings for the diagnosis and procedure codes from a contrastive machine-learning model trained using labeled valid and invalid diagnosis-procedure code combinations; determining a similarity-based compatibility measure for the diagnosis and procedure codes using the embeddings; aggregating the compatibility measure across multiple diagnosis codes or multiple procedure codes associated with the encounter using multi-code weighting logic; assigning a preliminary classification to the encounter based on comparison of the aggregated compatibility measure to at least one threshold, the preliminary classification indicating valid, invalid, or needs review; retrieving one or more current coding guidance passages corresponding to the diagnosis and procedure codes; confirming the preliminary classification when the retrieved one or more current coding guidance passages are consistent with the preliminary classification, or revising or overriding the preliminary classification when the retrieved one or more current coding guidance passages conflict with the preliminary classification, to produce a final classification; generating a policy-backed explanation using the retrieved coding guidance passages; and presenting, before claim submission, the final classification together with the policy-backed explanation.

3. A non-transitory computer-readable medium storing instructions that, when executed by one or more processors, cause the one or more processors to perform operations comprising:
receiving diagnosis and procedure code information for a healthcare encounter during a point-of-coding workflow; obtaining embeddings for the diagnosis and procedure code information from a contrastive machine-learning model trained on labeled valid and invalid diagnosis-procedure code combinations; determining an embedding-based compatibility measure for the diagnosis and procedure code information using a similarity computation applied to the embeddings;
applying multi-code weighting to the diagnosis and procedure code information to produce an aggregated compatibility determination; comparing the aggregated compatibility determination to at least one threshold to assign a preliminary classification indicating valid, invalid, or needs review; retrieving policy guidance relevant to the diagnosis and procedure code information;
confirming the preliminary classification when the retrieved policy guidance is consistent with the preliminary classification, or revising or overriding the preliminary classification when the retrieved policy guidance conflicts with the preliminary classification, to produce a final classification; generating a policy-cited explanation corresponding to the final classification; and
outputting the final classification and the policy-cited explanation during the point-of-coding workflow and before claim submission.

4. The system of claim 1, wherein the contrastive machine-learning model comprises a two-tower architecture in which a first tower generates embeddings for diagnosis codes and a second tower generates embeddings for procedure codes.

5. The system of claim 4, wherein the two-tower architecture is trained using a contrastive loss function that reduces the distance between embeddings of valid diagnosis-procedure code combinations and increases the distance between embeddings of invalid diagnosis-procedure code combinations.

6. The system of claim 1, wherein the contrastive machine-learning model is trained using labeled valid and invalid diagnosis-procedure code pairs, each pair derived from audited real-world coding and billing scenarios and associated with references to official coding guidance from one or more authoritative sources.

7. The system of claim 1, wherein the embeddings are precomputed during an offline training phase and stored in a format enabling retrieval during real-time validation without re-executing the contrastive machine-learning model for each validation request.

8. The system of claim 7, wherein the precomputed embeddings are stored in an HDF5-based storage format.

9. The system of claim 1, wherein the multi-code weighting assigns different weights to primary and secondary diagnosis codes when generating the aggregated compatibility determination.

10. The system of claim 1, wherein the preliminary classification comprises a three-class output indicating valid, invalid, or needs review, and the needs review classification is assigned when the aggregated compatibility determination falls within a predefined threshold band or when retrieved policy or guidance passages conflict with the preliminary classification.

11. The system of claim 1, wherein producing the final classification comprises:
confirming the preliminary classification when the retrieved one or more policy or guidance passages are consistent therewith; and revising or overriding the preliminary classification when the retrieved one or more policy or guidance passages conflict therewith.

12. The system of claim 1, wherein the one or more policy or guidance passages include at least one of: payer-specific guidance, guidance from the Centers for Medicare and Medicaid Services, guidance from the National Correct Coding Initiative, or internal compliance guidance.

13. The system of claim 1, wherein the explanation includes one or more citations to specific policy or guidance passages from which the explanation was generated.

14. The system of claim 1, wherein the system comprises a front-end user interface component and a backend validation service exposed through one or more network-accessible application programming interfaces, the front-end user interface component configured to receive the set of medical codes from a user and to display the final classification and the explanation to the user.

15. The system of claim 1, wherein the one or more processors are further configured to receive user feedback regarding a classification result and store the user feedback in a feedback data store for subsequent use in refining the contrastive machine-learning model or the policy guidance data.

16. The system of claim 15, wherein the stored user feedback is incorporated into a periodic retraining pipeline that updates the contrastive machine-learning model, the embeddings, or both based at least in part on the stored user feedback.

17. The system of claim 1, wherein the system is deployed as a containerized microservice configured for integration with one or more of: an electronic health record system, a practice management system, or a revenue cycle management platform.

18. The system of claim 1, wherein the policy guidance data is maintained in a retrieval index that is updated to reflect changes in coding rules, payer rules, or regulatory guidance, and wherein the one or more processors retrieve the one or more policy or guidance passages from the updated retrieval index.

19. The method of claim 2, wherein aggregating the compatibility measure comprises generating a single aggregated compatibility score for the encounter from individual compatibility measures computed for respective diagnosis-procedure code pairings within the encounter.

20. The method of claim 2, wherein retrieving the one or more current coding guidance passages is performed by querying an indexed collection of coding policies and guidelines to identify relevant guidance passages, and providing the identified passages as context for generating the policy-backed explanation.

21. The system of claim 1, wherein the one or more processors are further configured to employ approximate nearest neighbor indexing to accelerate retrieval of the embeddings, retrieval of the one or more policy or guidance passages, or both.
